(19) United States
(12) Patent Application Publication
(10) Pub. No.: US 2005/0019298 A1
(43) Pub. Date: Jan. 27, 2005

(54) Transfer-resistant cosmetic compositions

(75) Inventors: Haimanot Bekele, Baltimore, MD (US); George Endel Deckner, Cincinnati, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/824,298

(22) Filed: Apr. 14, 2004

Related U.S. Application Data
(60) Provisional application No. 60/462,864, filed on Apr. 14, 2003.

(51) Int. Cl. A61K 7/04; A61K 7/06; A61K 7/11

(52) U.S. Cl. 424/70.16

(57) ABSTRACT

The cosmetic compositions of the present invention provide a durable film after application that resists degradation over time. Cosmetic compositions of the present invention comprise: a) polymers selected from the group consisting of hydrophilic copolymers and terpolymers; b) Glycerols selected from the group consisting of glycerol, modified glycerols, and mixtures thereof; and c) a polar volatile solvent.

### CROSS REFERENCE TO RELATED APPLICATION

This application claims the benefit under 35 USC 119(e) to U.S. Application Ser. No. 60/462,864, filed Apr. 14, 2003.

### TECHNICAL FIELD

The present invention relates to transfer-resistant cosmetic compositions that comprise a hydrophilic copolymer or terpolymer in combination with glycerol or its derivatives and a polar volatile solvent. Upon application, the composition forms a thin, but durable film resistant to transfer upon contact with objects such as clothing, towels, cups, handkerchiefs and tissues. The present invention also relates to methods of enhancing performance and/or appearance in long wearing cosmetic products.

### BACKGROUND

Transfer resistant cosmetic compositions, particularly lip products, have gained increasing popularity over the last decade as consumers find themselves burdened with the stress of daily activities. These types of products allow consumers to apply the lip product fewer times in a day but still achieve a freshly applied appearance. One shortcoming of a number of these products, however, has been the undesirable feel and quality of wear on the lips that accompanies the use of such products. For example, consumers complain of a tight feeling on the lips that typically results from the inclusion of film forming agents into these products that provide the long wear characteristic. Additionally, consumers complain of the quality of wear of the lip color indicating that the color does not last throughout the day, i.e., the color does not retain a freshly applied, consistent look over an extended period of time. Thus, there remains a need for a lip product that not only provides a more pleasurable feel when applied and worn on the lips but, at the same time, provides a long, fresh-look wear for the consumer. Compositions of the present invention not only provide profound film resistant to transfer upon contact with objects such as clothing, towels, cups, handkerchiefs and tissues throughout the day, but also provides the qualities to maintaining a freshly applied, consistent look. Additionally, appearance benefits such as gloss and shine are exemplified in a single step application of the invention.

### SUMMARY OF THE INVENTION

The cosmetic compositions of the present invention provide a durable film after application that resists degradation over time. Cosmetic compositions of the present invention comprise:
a) polymers selected from the group consisting of hydrophillic copolymers and terpolymers;
b) Glycerols selected from the group consisting of glycerol, modified glycerols, and mixtures thereof; and
c) polar volatile solvent.

### DETAILED DESCRIPTION

The compositions of the present invention are transfer-resistant cosmetic compositions, particularly lip products. Lip products of the present invention exhibit a viscosity of from about 500 cP to about 15,000 cP, preferably from about 500 cP to about 8,000 cP, more preferably from about 1,000 cP to about 5,000 cP at room temperature.

Applicants have found that cosmetic compositions as detailed herein are particularly resistant against insult when subjected to the typical rigors of daily exposure.

As used herein, “comprising” means that other steps and ingredients can be added. This term encompasses the terms “consisting of” and “consisting essentially of”. The phrase “consisting essentially of” means that the composition may include additional ingredients, but only if the additional ingredients do not materially alter the basic and novel characteristics of the claimed compositions or methods.

All percentages, parts and ratios are based upon the total weight of the topical compositions of the present invention and all measurements made are at 25° C., unless otherwise specified. All such weights as they pertain to listed ingredients are based on the active level and, therefore, do not include carriers or by-products that may be included in commercially available materials, unless otherwise specified.

All publications cited herein are hereby incorporated by reference in their entirety.

As used herein, “lip product” means a cosmetic that can be applied to the lips and may be in the form of a liquid, conventional bullet, gel, cream, lip color pen, and the like.

The compositions of the present invention necessarily comprise the following ingredients.

Copolymer and Terpolymer

Compositions of the present invention comprise polymers selected from the group consisting of hydrophilic copolymers and terpolymers. These copolymers and terpolymers are such that they are soluble or dispersible in polar solvents and are added to the composition at levels of from about 1% to about 50%, preferably from about 1% to about 30%, more preferably from about 5% to about 20%, and most preferably from about 8% to about 15%. In a preferred embodiment of the invention, the copolymers 2-hydroxyethyl methacrylate (HEMA) and 4-hydroxybutyl acrylate (HBA) are used. Copolymers useful in the present invention are prepared according to the methods described in US 2002037006. Suitable copolymers have the following general structure: 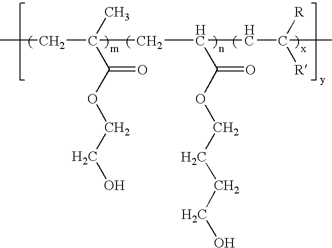
R=CH₃ or H
R′=aliphatic, aromatic, carboxy acids, carboxy esters, amides, hydroxyl, amines

In the present invention, m, n, and y are from about 1 to about 100, and x is from about 0 to 100. Furthermore, in copolymers of 2-hydroxyethyl methacrylate (HEMA) and 4-hydroxybutyl acrylate (HBA), the ratio of m to n is from about 0.7:1 to about 4:1, preferably from about 1:1 to about 2.5:1, and most preferably about 1.5:1.

The glass transition of the copolymers and terpolymers useful in the present invention are from about 5 degrees to about 55° C., preferably from about 15° C. to about 30° C.

Additionally, compositions of the present invention may comprise silicone modified fluorinated polymers used alone or in combination with the copolymers disclosed above.

Glycerols

Compositions of the present invention may contain glycerols selected from the group consisting of glycerols, modified glycerols, and mixtures thereof. Glycerols of the present invention improve the performance of the polymer films allowing it to be more flexible, wear resistant and adhesive to skin, lips, hand and body including, but not limited to, glycerol, glycerol esters, and glycerol ethers. Examples of modified glycerols are ethoxylated glycerols such as glycereth G-26, glycereth G-7. Other examples of modified glycerols are glycerol esters such as triglycerides.

Other ingredients useful in the present invention that modify the film properties include, but are not limited to, rosin esters and gylceryl ethers, oleic acid, octyldodecyl myristate, polyalkylene oxide and the like.

Volatile Carrier

In the present invention, it is necessary that the copolymer or terpolymer be incorporated into a carrier, specifically a volatile carrier which quickly volatilizes from the surface of the lips leaving the above-discussed thin-durable film. The volatile carrier must solubilize or disperse the copolymer or terpolymers.

The volatile carrier comprises from about 50% to about 95%, preferably from about 50% to about 80%, and most preferably from about 50% to about 70% of the composition. The volatile carrier of the present invention can be water, alcohols, or mixtures thereof. Suitable alcohols include ethyl alcohol, isopropyl alcohol, butyl alcohol or other suitable alkyl alcohols. In a preferred embodiment, the volatile carrier is a mixture of water and alcohol wherein water is from about 30% to about 90%, more preferably from about 50% to about 80%. Other useful mixtures of solvents are selected from a group consisting of water, alcohols, propylene glycols, ethylene glycols, suitable aliphatic ethers, suitable alphatic esters, and mixtures thereof.

Thickeners/Structure Builders

The cosmetic composition of the present invention may also comprise at least one of the group consisting of thickeners or structure builders which have the function of dispersing pigments in addition to building viscosity. Thickeners and/or structure builders useful for the present invention include, but are not limited to, organically modified clays, fumed silica, silicone polyamide, and polymeric thickeners. Examples of useful polymeric thickeners include, but are not limited to, diblock/triblock copolymers, random copolymers and silsequioxane crosspolymers. Particularly useful are polyacrylamide, polysaccharide, polyacrylate, and cationic polyacrylate. Organically modified clays useful for the present invention include, but are not limited to, hectorite, bentonite, smectite and montmorillonite clay.

When thickeners and/or structure builders are present, the compositions comprise less than about 10%, by weight of the composition, more preferably, less than about 5%, and most preferably from about 0.1% to about 3%.

Pigments

Pigments suitable for use herein are all inorganic and organic colors/pigments suitable for use in lip composition compositions. These are usually aluminum, barium or calcium salts or lakes. Lakes are either a pigment that is extended or reduced with a solid diluent or an organic pigment that is prepared by the precipitation of a water-soluble dye on an adsorptive surface, which usually is aluminum hydrate. A lake also forms from precipitation of an insoluble salt from an acid or basic dye. Calcium and barium lakes are also used herein.

Preferred lakes of the present invention are Red 3 Aluminum Lake, Red 21 Aluminum Lake, Red 27 Aluminum Lake, Red 28 Aluminum Lake, Red 33 Aluminum Lake, Yellow 5 Aluminum Lake, Yellow 6 Aluminum Lake, Yellow 10 Aluminum Lake, Orange 5 Aluminum Lake and Blue 1 Aluminum Lake, Red 6 Barium Lake, Red 7 Calcium Lake, Red 30 Talc Lake, and Red 30 Aluminum Lake.

Other colors and pigments can also be included in the lip compositions, such as dyes and pearls, titanium oxides, Red 6, Red 21, Blue 1, Orange 5, and Green 5 dyes, chalk, talc, iron oxides and titanated micas.

Compositions of the present invention contain sufficient pigments to provide the look sought by the user. The pigments are used herein at levels relative to the level of the diorganopolysiloxane polymers disclosed above. This level is expressed as a ratio of the combination of diorganopolysiloxane polymer and organosiloxane resin to pigment. In the present invention this ratio is from about 1:1 to about 30:1, preferably from about 1.5:1 to about 15:1, and most preferably from about 2:1 to about 10:1.

Waxes

Waxes may be used in the present invention provided they are used at levels which do not interfere with film formation process.

Waxes are defined as lower-melting organic mixtures or compounds of high molecular weight, solid at room temperature and generally similar in composition to fats and oils except that they contain no glycerides. Some are hydrocarbons, others are esters of fatty acids and alcohols. Waxes useful in the present invention are selected from the group consisting of animal waxes, vegetable waxes, mineral waxes, various fractions of natural waxes, synthetic waxes, petroleum waxes, ethylenic polymers, hydrocarbon types such as Fischer-Tropsch waxes, silicone waxes, and mixtures thereof.

The specific waxes useful in the present invention are selected from the group consisting of synthetic waxes, ozokerite, jojoba esters, “Unilins”, available from Petrolite Corporation, “Ganex” alkylated polyvinylpyrrolidines available from the ISP Company, fatty alcohols from C22 to C50 and mixtures thereof. Synthetic waxes include those disclosed in Warth, Chemistry and Technology of Waxes, Part 2, 1956, Reinhold Publishing; herein incorporated by reference. The waxes most useful herein are selected from the C₈ to C₅₀ hydrocarbon waxes. Such waxes include long chained polymers of ethylene oxide combined with a dihydric alcohol, namely polyoxyethylene glycol. Such waxes include Carbowax™ available from Carbide and Carbon Chemicals company. Other synthetic waxes include long-chained polymers of ethylene with OH or other stop length grouping at end of chain. Such waxes include the Fischer-Tropsch waxes as disclosed in the text disclosed above at pages 465-469 and include Rosswax, available from Ross company and PT-0602 available from Astor Wax Company.

The cosmetic compositions of the present invention are also in the form of liquid lip products. As such, the compositions comprise less than about 2%, by weight of the composition, of waxes, more preferably, less than about 1%, and most preferably less than about 0.5%.

Other Ingredients

There are a number of other ingredients approved for use in the cosmetic art that may be used in compositions of the present invention. Such ingredients are those approved for use in cosmetics and can be found listed in reference books such as the CTFA Cosmetic Ingredient Handbook, Second Edition, The Cosmetic, Toiletries, and Fragrance Association, Inc. 1988, 1992. Said materials may be used provided their inclusion does not significantly disrupt the composition once it has been applied wherein a film has been formed. Said ingredients include fragrances, flavor oils, skin care ingredients such as sunscreen, emulsifiers and the like. Hypoallergenic compositions can be made into the present invention where said compositions do not contain fragrances, flavor oils, lanolin, sunscreens, particularly PABA, or other sensitizers and irritants.

Complementary products may be used in conjunction with the present invention to complement the composition and improve its aesthetic appeal to the user. In particular, it is intended that the cosmetic compositions of the present invention may be paired in a lip cosmetic kit with such a complementary product.

It is specifically envisioned that the complementary products used in the present invention manner wherein such a product is applied over the film formed after application of the cosmetic composition of the present invention. For example in the case of lip compositions, a complementary product may be utilized to enhance the gloss and shine of the lips and provide a lubricious feeling. Such products, otherwise known as an “overcoat” or “topcoat” may be in a stick or liquid form and can include any that are commercially available or to be developed, provided the aggregate of the materials comprising the overcoat does not significantly disrupt the composition of the present invention. The overcoat compositions may be clear or transparent or may contain dyes and/or colorants that when viewed along with the overcoat, produce a desired color.

One such material that has been shown to be quite useful in formulating complementary products is polyol polyesters, such as sucrose polyesters (herein referred to as SPE'S). SPE's are synthesized molecules derived from sugar and vegetable oil and have been extensively disclosed in the patent literature in context of a non-digestible oils. Such compositions are generally disclosed in U.S. Pat. No. 3,600,186, issued Aug. 17, 1971; U.S. Pat. No. 4,005,195, issued Jan. 25, 1977; U.S. Pat. No. 4,005,196, issued Jan. 25, 1977; all assigned to the Procter & Gamble Company and all herein incorporated by reference.

It has found that overcoat compositions having a significant level of SPE'S are incompatible with the lip composition of the present composition wherein upon their application, the cosmetic composition of the present invention is not disrupted.

In a preferred embodiment of the present invention, a complementary product comprising a topcoat composition comprises a safe and effective amount of a polymeric vegetable oil emollient. More preferably, the polymeric vegetable oil emollient is a copolymer of Brassica Campestris and Aleurites Fordii oil (also known as polytriglyceryl erucate/eliostearate), which is an unsaturated complex ester ranging in molecular weight from 25,000 to 110,000 (or 6500 to about 7500 daltons). This emollient is commercially available from Tri-K under the tradename Glossamer L-6600 and allows the topcoat to form a protective film over the cosmetic compositions of the present invention. Such a topcoat composition aids in providing a creamier feel to the applied basecoat and topcoat and also enhances the water and rub-off resistance of the cosmetic composition as well as the topcoat composition. In more preferred embodiments, the polymeric vegetable oil emollient is present in an amount of from about 0.1% to about 50%, more preferably, from about 2% to about 20%, and most preferably, from about 4% to about 15%, by weight of the topcoat composition.

Copolymers of the present invention may also be used as topcoats. Additionally, silicone modified fluorinated polymers and modified silicones selected from the group consisting of aminosilicones, carboxy modified silicones, epoxy modified silicones, and mixtures thereof may be used alone or in combination with the copolymers of the present invention as topcoats.

### METHOD OF USING THE INVENTION

The method of the present invention is straightforward. The user applies the composition of the present invention from a suitable liquid cosmetic applicator directly onto the skin. One such applicator used for liquid lip products is a liquid pen package disclosed in British Patent 21198037, issued May 9, 1990, assigned to Mitsubishi Pencil Co., Ltd. of Japan. An alternative package is one where a wand is dipped into a reservoir wherein the composition on the tip of the wand is applied to the skin surface. Such packages are disclosed in Japanese Utility Model 64 000822 Y2, to Shiseido.

Another cosmetic dispenser that is useful for the present invention is a unidirectional twist-up dispensing device with incremental dosing as disclosed in U.S. Pat. No. 5,851,079, issued on Dec. 22, 1998 to Richard L. Horstman et al. Such a twist-up dispensing device can include a hollow housing defining a chamber having an open dispensing end and a piston located within the chamber being limited to translational movement within the chamber. The piston preferably having a threaded rod extending therefrom that engages with a threaded aperture in an actuator such that advancement of the piston toward the dispensing end occurs when the actuator is rotated. Rotation of the actuator causes the product to be dispensed from the dispensing end. An applicator is preferably attached to the dispensing end of the housing in fluid communication with the chamber wherein the product is dispensed through the applicator. The applicator can comprise a ferrule and an application portion wherein the ferrule is attached to the dispensing end of the housing and the application portion has at least one orifice located therein. Several versions of applicators can be utilized including, for example, a fiber brush or an application surface having flocking thereon. Flocking is a mat of thin, short, plastic fibers substantially perpendicular to the application surface. The bristles of a fiber brush are preferably tapered and made of a plastic material. Alternatively, the user may use a more traditional applicator or implement known in the art.

As stated above, the user applies the composition wherein the user allows the composition to dry before subjecting the composition to insult. Once the composition is dried, a complimentary product such as the topcoat product disclosed above may be applied over the dried product to provide the user with an aesthetically pleasing affect. Topcoat compositions may utilize the same dispensing device as described for use of the compositions of the present invention.

### EXAMPLES

The following example illustrates the claimed cosmetic compositions of the present invention but are not intended to be limiting thereof:

### EXAMPLE

| Ingredient | Conc. (%) |
|---|---|
| PolyHEMA/HBA | 10.0 |
| TiO2 Anatase | 2.24 |
| Yellow 5 Al Lake | 0.8 |
| Red 7 Ca Lake | 1.28 |
| Blue #1 Al Lake | 0.08 |
| Red Iron Oxide | 0.96 |
| Silk Mica | 2.64 |
| Propylparaben | 0.2 |
| Salcare SC-95 | 0.99 |
| Ethosperse | 8.0 |
| Ethanol | 21.84 |
| Water | 50.97 |
| Total | 100 |

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A cosmetic composition comprising:
a) polymers selected from the group consisting of hydrophilic copolymers and terpolymers;
b) Glycerols selected from the group consisting of glycerol, modified glycerols, and mixtures thereof; and
c) a polar volatile solvent.

2. The cosmetic composition of claim 1 wherein the polymer is a hydrophilic copolymer.

3. The cosmetic composition of claim 2 wherein the hydrophilic copolymer is poly(2-hydroxyethyl methacrylate-co-4-hydroxybutyl acrylate).

4. The cosmetic composition of claim 3 wherein the hydrophilic copolymer is present at a level of from about 1% to about 50%.

5. The cosmetic composition of claim 3 wherein the HEMA/HBA ratio is from about 0.7:1 to about 4:1 and the glass transition is from about 5° C. to about 55° C.

6. The cosmetic composition of claim 1 further comprising a polymeric thickener.

7. The cosmetic composition of claim 6 wherein the polymeric thickener is cationic polyacrylate.

8. The cosmetic composition of claim 1 wherein the glycerol is glyceryl ether.

9. The cosmetic composition of claim 1 wherein the polar volatile solvent is a mixture of water and ethanol.

10. The cosmetic composition of claim 9 comprising about 70% water.

11. A lip product comprising the composition of claim 1 having a viscosity of from about 500 cP to about 15,000 cP at room temperature.

12. A method of applying the composition of claim 1 to lips, said method comprising the steps of:
a. applying the composition from a suitable cosmetic applicator directly onto the lips; and
b. allowing said composition to form a dry film on the lips before subjecting the film to insult.

13. The method of claim 12 that comprises the additional step of applying a complimentary product comprising a topcoat composition on said dried film where upon applying said complimentary product said film is not disrupted.

14. The method of claim 13 wherein said topcoat composition comprises a safe and effective amount of a polymeric vegetable oil emollient.

15. The method of claim 13 wherein said topcoat composition comprises a safe and effective amount of a silicone selected from the group consisting of silicone modified fluorinated polymers and modified silicones selected from the group consisting of aminosilicones, carboxy modified silicones, epoxy modified silicones, and mixtures thereof.

16. A lip cosmetic kit comprising:
(I) a cosmetic composition comprising:
a) polymers selected from the group consisting of hydrophilic copolymers and terpolymers;
b) Glycerols selected from the group consisting of glycerol, modified glycerols, and mixtures thereof; and
c) a polar volatile solvent; and
(II) a complimentary product comprising a topcoat composition that comprises a safe and effective amount of a polymeric vegetable oil emollient.

17. A lip cosmetic kit comprising:
(I) a cosmetic composition comprising:
a) polymers selected from the group consisting of hydrophilic copolymers and terpolymers;
b) Glycerols selected from the group consisting of glycerol, modified glycerols, and mixtures thereof; and
c) a polar volatile solvent; and
(II) a complimentary product comprising a topcoat composition that comprises a topcoat composition that comprises a safe and effective amount of silicone modified fluorinated polymers.
